(19) United States
(12) Patent Application Publication
(10) Pub. No.: US 2026/0262696 A1
(43) Pub. Date: Sep. 10, 2026

(54) DIETARY FIBER, PREPARATION METHOD AND USE THEREOF

(71) Applicant: ZheJiang University ZhongYuan Institute, Zhengzhou City (CN)

(72) Inventors: Ebenezer Ola FALADE, Zhengzhou City (CN); Xingqian YE, Zhengzhou City (CN); Kouadio Jean Eric-Parfait KOUAMÉ, Zhengzhou City (CN); Yunyun ZHENG, Zhengzhou City (CN); Bingge LIU, Zhengzhou City (CN)

(21) Appl. No.: 19/335,568

(22) Filed: Sep. 22, 2025

(30) Foreign Application Priority Data
Mar. 7, 2025 (CN) 202510264688.2

(51) Int. Cl. A23L 33/24; C12N 9/02; C12P 19/04

(52) U.S. Cl. CPC A23L 33/24; C12N 9/0061; C12P 19/04; C12Y 110/03002

(57) ABSTRACT

A dietary fiber, preparation method and the use thereof, which belong to the technical field of foods. The preparation method does not require the use of a chlorine-containing reagent and/or ozone, and includes the steps of mixing lignocellulosic biomass with a laccase reagent for an enzyme treatment to obtain an enzyme-treated fiber; mixing the enzyme-treated fiber with a bleaching agent for bleaching treatment under ultrasonic and microwave conditions to obtain a bleached fiber, where the bleaching agent is an aqueous solution containing hydrogen peroxide and an acid reagent; mixing the bleached fiber with an alkaline solution for an alkaline treatment under ultrasonic and microwave conditions to obtain an alkali-treated slurry; and mixing the alkali-treated slurry with aqueous hydrogen peroxide solution for an oxidation treatment to obtain the dietary fiber.

### CROSS REFERENCE TO RELATED APPLICATION

This patent application claims the benefit and priority of Chinese Patent Application No. 202510264688.2 filed with the China National Intellectual Property Administration on Mar. 7, 2025, the disclosure of which is incorporated by reference herein in its entirety as part of the present application.

### TECHNICAL FIELD

The present disclosure relates to the technical field of foods, and to dietary fiber, and preparation method and the use thereof.

### BACKGROUND

Brewer's spent grain (BSG) is a valuable source of dietary fiber, which contains arabinoxylan and beta-glucan, with significant functional advantages for the applications in food and nutrient products. However, the presence of lignin and other chromophoric compounds typically render BSG fibers brown, which limit their applications in foods and nutrient products.

The traditional fiber bleaching methods often require the use of a chlorine-containing reagent or ozone. However, these methods often fail to achieve the desired whiteness of dietary fibers and damage the structural integrity of fibers. At the same time, chlorine-containing reagents and ozone may produce toxic by-products that harm the environment, and require complex waste management procedures, thus restricting their use in the preparation of dietary fibers. Hydrogen peroxide (H₂O₂) is an environmentally friendly bleach that decomposes into water and oxygen and is environmentally friendly. However, if only hydrogen peroxide is used as bleach, it is difficult to achieve the desired whiteness of the dietary fiber.

### SUMMARY

An objective of the present disclosure is to provide dietary fiber, and a preparation method and the use thereof. The dietary fiber prepared by the method of the present disclosure has high whiteness and good structural integrity, and the preparation process is environmentally friendly.

The present disclosure provides a method for preparing a dietary fiber without using a chlorine-containing reagent and/or ozone, including the steps of:
mixing lignocellulosic biomass with a laccase reagent for enzyme treatment to obtain an enzyme-treated fiber;
mixing the enzyme-treated fiber with a bleaching agent for a bleaching treatment under ultrasonic and microwave conditions to obtain a bleached fiber, where the bleaching agent is an aqueous solution containing hydrogen peroxide and an acid reagent;
mixing the bleached fiber with an alkaline solution for an alkaline treatment under ultrasonic and microwave conditions to obtain an alkali-treated slurry; and,
mixing the alkali-treated slurry with aqueous hydrogen peroxide solution for an oxidation treatment to obtain dietary fiber.

In a preferred embodiment, the lignocellulosic biomass includes at least one selected from the group consisting of brewer's spent grain, sugar cane bagasse, rice hulls and straws.

In a preferred embodiment, the laccase reagent includes laccase and a buffer, the laccase has an activity of 180-250 U/g, and the buffer has a pH of 4.5-5.5; and the concentration of the laccase in the laccase reagent is 13.5-16.5 U/g.

In a preferred embodiment, the mass ratio of the lignocellulosic biomass to the laccase reagent is 13-14:1; and the enzyme treatment is carried out at a temperature of 35-50° C. for 6-14 h.

In a preferred embodiment, the concentration of hydrogen peroxide in the bleaching agent is 2-3 wt %, the concentration of the acid reagent in the bleaching agent is 2-5 wt %, and the acid reagent is acetic acid; and the ratio of the amount of the enzyme-treated fiber to the amount of the bleaching agent is 40 g:280-320 mL; and the bleaching treatment is carried out under conditions including: an ultrasonic frequency of 60-80 kHz, a microwave power of 500-700 W, a temperature of 40-70° C., and a duration of 8-20 min.

In a preferred embodiment, the alkaline solution is selected from the group consisting of a NaOH solution and a Na₂CO₃ solution, the concentration of the NaOH solution is 0.5-1 wt %, and the concentration of the Na₂CO₃ solution is 1.5-2 wt %; the ratio of the amount of the bleached fiber to the amount of the alkaline solution is 1-2 g:7.5-10 mL; and the alkaline treatment is carried out under conditions including: an ultrasonic frequency of 60-90 kHz, a microwave power of 500-700 W, a temperature of 70-80° C., and a duration of 20-30 min.

In a preferred embodiment, the concentration of the aqueous hydrogen peroxide solution is 2-4 wt %, and the ratio of the amount of the bleached fiber to the amount of the aqueous hydrogen peroxide solution is 40 g:280-320 mL; and the oxidation treatment includes a first-stage oxidation treatment and a second-stage oxidation treatment in sequence; the first-stage oxidation treatment is carried out under ultrasonic and microwave conditions, the conditions of the first-stage oxidation treatment including: an ultrasonic frequency of 60-90 kHz, a microwave power of 500-700 W, a temperature of 40-70° C., and a duration of 10-20 min; and the second-stage oxidation treatment is carried out under conditions including: a temperature of 20-30° C., and a duration of 1.5-2.5 h.

The present disclosure provides a dietary fiber prepared by the preparation method described in the technical solution.

In a preferred embodiment, the dietary fiber has a CIE whiteness index of 77-86%.

The present disclosure provides the use of the dietary fiber described in the technical solution in food or nutrient products.

An object of the invention is to provide an environmentally friendly bleaching method for insoluble dietary fiber (IDF) in lignocellulosic biomass. Specifically, in the present disclosure, laccase is used for the enzyme treatment of the lignocellulosic biomass (e.g., brewer's spent grain, etc.), which may oxidize and decompose lignin, making it easier to remove. In the present disclosure, an aqueous solution containing hydrogen peroxide and an acid reagent is used as a bleaching agent, and lignin may be further degraded by bleaching under ultrasonic and microwave conditions, which is beneficial for improving the bleaching effect and ensuring the structural integrity of the fiber. In particular, ultrasonic waves may produce local agitation and cavitation phenomena to form a high-energy microenvironment, promoting the penetration of the bleaching agent into the fiber matrix, accelerating chemical reactions, and facilitating the decomposition of lignin and other chromophoric compounds; and microwaves may provide fast and uniform heating, which facilitates accelerated chemical reactions between the bleaching agent and the lignin and other chromophoric compounds, thereby shortening the overall processing time and reducing energy consumption. The present disclosure involves alkaline treatment under ultrasonic and microwave conditions and then an oxidation treatment, which are conducive to further improving the bleaching effect based on ensuring the integrity of the fiber structure. The method provided by the present disclosure does not require the use of a chlorine-containing reagent and/or ozone and thus is environmentally friendly, and the resulting dietary fiber is suitable for use in food or nutrient products.

Unexpectedly, the dietary fiber prepared by the present method not only achieves a CIE whiteness index of 77-86% but also retains a high proportion of arabinoxylan-derived monosaccharides (arabinose and xylose) and their characteristic glycosidic linkages. Such preservation of hemicellulose structure, confirmed by monosaccharide and linkage analyses, distinguishes the disclosed fiber from those obtained by conventional chlorine or single-step peroxide bleaching.

These and other objects, features, and advantages of the present invention will become readily apparent upon a review of the following detailed description of the present invention, in view of the drawings and appended claims.

### BRIEF DESCRIPTION OF THE DRAWINGS

FIG. 1 shows a flow chart of the preparation of a dietary fiber in an embodiment of the present disclosure;

FIGS. 2A-2G show physical pictures of the products prepared in Examples 1-3 and Comparative examples 1-3;

FIG. 3 shows Fourier-transform infrared spectra of the products prepared in Examples 1-3 and Comparative example 1;

FIG. 4 shows X-ray diffraction spectra of the products prepared in Examples 1-3 and Comparative example 1;

FIG. 5 shows TGA curves of the products prepared in Examples 1-3 and Comparative example 1;

FIG. 6 shows DSC curves of the products prepared in Examples 1-3 and Comparative example 1;

FIGS. 7A-7E show SEM images of the products prepared in Examples 1-3 and Comparative example 1;

FIGS. 8A-8D are LC-MS/MS chromatograms illustrating the separation of standard monosaccharides [labelled as (A)] and example 1 monosaccharides [labelled as (B)], all identified in accordance with their retention time;

FIG. 9 shows Monosaccharide composition (%) of dietary fibers from brewer's spent grain subjected to different delignification treatments: enzymatic pretreatment alone (comparative example 1), example 1, comparative example 3, and untreated BSG fiber;

FIG. 10 shows differences in retention of major arabinoxylan glycosidic linkages in dietary fibers from brewer's spent grain (BSG) subjected to different delignification treatments: untreated BSG fiber, example 1 and comparative example 3; and,

FIGS. 11A-11C show the MRM chromatograms showing linkage analysis for (A) Comparative example 3 (B) Example 1 and (C) Untreated BSG. The short dotted blue trace represents transitions of the quantifying ion at m/z 175.1, whereas the solid red and green traces represent transitions of qualifying ions at m/z 231.2 and m/z 217.1, respectively.

### DETAILED DESCRIPTION OF THE EMBODIMENTS

At the outset, it should be appreciated that like drawing numbers on different drawing views identify identical, or functionally similar, structural elements. It is to be understood that the claims are not limited to the disclosed aspects.

Furthermore, it is understood that this disclosure is not limited to the particular methodology, materials and modifications described and as such may, of course, vary. It is also understood that the terminology used herein is for the purpose of describing particular aspects only and is not intended to limit the scope of the claims. Those in the art will understand that any suitable material, now known, or hereafter developed, may be used in forming the present invention described herein.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments.

The word “example” or “exemplary” is used herein to mean “serving as an example, instance, or illustration.” Any implementation described herein as “exemplary” is not necessarily to be construed as preferred or advantageous over other implementations.

If the specification states a component or feature “may,” “can,” “could,” “should,” “would,” “preferably,” “possibly,” “typically,” “optionally,” “for example,” “often,” or “might” (or other such language) be included or have a characteristic, that a specific component or feature is not required to be included or to have the characteristic. Such a component or feature may be optionally included in some embodiments, or it may be excluded.

It should be noted that the terms “including”, “includes”, “having”, “has”, “contains”, and/or “containing”, should be interpreted as being substantially synonymous with the terms “comprising” and/or “comprises”.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. The term “approximately” is intended to mean values within ten percent of the specified value.

It should be understood that use of “or” in the present application is with respect to a “non-exclusive” arrangement, unless stated otherwise. For example, when saying that “item x is A or B,” it is understood that this can mean one of the following: (1) item x is only one or the other of A and B; (2) item x is both A and B. Alternately stated, the word “or” is not used to define an “exclusive or” arrangement. For example, an “exclusive or” arrangement for the statement “item x is A or B” would require that x can be only one of A and B. Furthermore, as used herein, “and/or” is intended to mean a grammatical conjunction used to indicate that one or more of the elements or conditions recited may be included or occur. For example, a device comprising a first element, a second element and/or a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element.

Moreover, as used herein, the phrases “comprises at least one of” and “comprising at least one of” in combination with a system or element is intended to mean that the system or element includes one or more of the elements listed after the phrase. For example, a device comprising at least one of: a first element; a second element; and, a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element. A similar interpretation is intended when the phrase “used in at least one of” is used herein. The invention is an infant support mat that combines novel materials with a unique ergonomic geometry. In a preferred embodiment, the mat comprises a unitary core (although the core does not have to be unitary—it could be made of several segments) and a removable cover.

The present disclosure provides a method for preparing a dietary fiber without using a chlorine-containing reagent and/or ozone, including the steps of:
mixing lignocellulosic biomass with a laccase reagent for an enzyme treatment to obtain an enzyme-treated fiber;
mixing the enzyme-treated fiber with a bleaching agent for a bleaching treatment under ultrasonic and microwave conditions to obtain a bleached fiber, where the bleaching agent is an aqueous solution containing hydrogen peroxide and an acid reagent;
mixing the bleached fiber with an alkaline solution for an alkaline treatment under ultrasonic and microwave conditions to obtain an alkali-treated slurry; and,
mixing the alkali-treated slurry with aqueous hydrogen peroxide solution for an oxidation treatment to obtain the dietary fiber; wherein the method yields a dietary fiber having a CIE whiteness index of 77-86% and retaining arabinoxylan structure as evidenced by arabinose and xylose content and glycosidic linkages at levels greater than fibers produced by chlorine bleaching.

The process produces a bright white dietary fiber having a CIE whiteness index of 77-86% while unexpectedly preserving arabinoxylan structure, including arabinose and xylose content and key glycosidic linkages.

In the present disclosure, unless otherwise specified, the raw materials used are commercially available products well known to those skilled in the art or are prepared by methods well known to those skilled in the art.

In the present disclosure, lignocellulosic biomass is mixed with a laccase reagent for an enzyme treatment to obtain enzyme-treated fiber. As an embodiment of the present disclosure, the lignocellulosic biomass may include at least one selected from the group consisting of brewer's spent grain (BSG), sugar cane bagasse, rice hulls and straws, and the straws may be corn straw and/or wheat straw. In an embodiment of the present disclosure, brewers' spent grain is specifically used as an example for illustration. As an embodiment of the present disclosure, the lignocellulosic biomass is preferably dried, ground, and sieved sequentially before being subjected to an enzymatic treatment. The drying temperature may be 75-85° C. and specifically may be 80° C., and the drying time may be 10-15 h and specifically maybe 12 h. The sieved lignocellulosic biomass having a particle size of 50-100 m is subjected to subsequent enzyme treatment. The grinding and sieving are not particularly limited in the present disclosure, if it may be ensured that lignocellulosic biomass having a particle size that satisfies the requirements may be obtained.

As an embodiment of the present disclosure, the laccase reagent may include laccase and a buffer, the activity of the laccase may be 180-250 U/g, specifically may be 180 U/g, 190 U/g, 200 U/g, 210 U/g, 220 U/g, 230 U/g, 240 U/g or 250 U/g. The buffer may be a sodium acetate buffer, which may have a pH of 4.5-5.5, specifically 4.5, 4.8, 5, 5.2 or 5.5. For example, the concentration of the laccase in the laccase reagent may be 13.5-16.5 U/g BSG, and specifically may be 14 U/g BSG, 14.5 U/g BSG, 15 U/g BSG, 15.5 U/g BSG or 16 U/g BSG, based on BSG.

In the present disclosure, laccase was used for the enzyme treatment of the lignocellulosic biomass, which may effectively oxidize and decompose lignin and other non-cellulosic impurities. In the embodiments of the present disclosure, with the pH of the buffer being limited in the ranges mentioned above, it is beneficial to enable the laccase to have better activity and reduce the environmental impact.

As an embodiment of the present disclosure, the mass ratio of the lignocellulosic biomass to the laccase reagent may be 13-14:1, and specifically may be 13.33:1. As an embodiment of the present disclosure, the temperature of the enzyme treatment may be 35-50° C., and specifically may be 35° C., 37° C., 40° C., 42° C., 45° C., 47° C., or 50° C. The duration of the enzyme treatment may be 6-14 h, and specifically may be 6 h, 8 h, 10 h, 12 h or 14 h. In the embodiments of the present disclosure, the enzyme treatment under the conditions described above may increase fiber accessibility and allow for the full oxidative degradation of lignin, resulting in easier removal of the lignin. This step also exposes arabinoxylan chains, which is evidenced by increased arabinose/xylose retention in Example 1.

As an embodiment of the present disclosure, after the enzyme treatment, solid-liquid separation is further included; the solid-liquid separation method may be filtration, and the solid material obtained after the solid-liquid separation is the enzyme-treated fiber.

In the present disclosure, after obtaining the enzyme-treated fiber, the enzyme-treated fiber is mixed with a bleaching agent for a bleaching treatment under ultrasonic and microwave conditions to obtain a bleached fiber. In the present disclosure, the bleaching agent is an aqueous solution containing hydrogen peroxide and an acid reagent. In the present disclosure, an aqueous solution containing hydrogen peroxide and an acid reagent is used as a bleaching agent, and the bleaching treatment is carried out under ultrasonic and microwave conditions. With ultrasonic and microwave radiation, a combined action of cavitation and dielectric heating is produced, which may enhance chemical penetration, accelerate chemical reaction, and reduce processing time, thereby further promoting lignin decomposition and improving the bleaching effect.

As an embodiment of the present disclosure, the concentration of hydrogen peroxide in the bleaching agent may be 2-3 wt %, and specifically may be 2 wt %, 2.5 wt %, or 3 wt %; the concentration of the acid reagent in the bleaching agent may be 2-5% wt %, and specifically may be 2 wt %, 3 wt %, 4 wt % or 5 wt %; and the acid reagent may be acetic acid. In the present disclosure, the cooperative oxidation action of hydrogen peroxide and acetic acid produces peracetic acid, which may efficiently degrade remaining lignin and other chromophoric compounds, thereby achieving a significant bleaching effect.

As an embodiment of the present disclosure, the ratio of the amount of the enzyme-treated fiber to the amount of the bleaching agent may be 40 g:280-320 mL and specifically may be 40 g:300 mL. As an embodiment of the present disclosure, the bleaching treatment is carried out under conditions including: an ultrasonic frequency that may be 60-80 kHz, and specifically may be 60 kHz, 65 kHz, 70 kHz, 75 kHz or 80 kHz; a microwave power that may be 500-700 W, and specifically may be 500 W, 550 W, 600 W, 650 W, or 700 W; a temperature that may be 40-70° C., and specifically may be 40° C., 50° C., 60° C., or 70° C.; and the duration of the bleaching treatment that may be 8-20 min, and specifically may be 8 min, 10 min, 15 min, or 20 min. In an embodiment of the present disclosure, the bleaching treatment is carried out under the conditions described above; the cavitation phenomenon generated by ultrasonic waves may maximize the permeability of the bleaching agent, and simultaneously microwaves are used to maintain a stable temperature and increase chemical reactivity, thus avoiding damage to the integrity of the fiber structure while ensuring a better bleaching effect; moreover, it also helps reduce reagent amount and energy consumption, enhancing the eco-friendliness of the preparation process.

As an embodiment of the present disclosure, after the bleaching treatment, the preparation method further includes: subjecting the system obtained after the bleaching treatment to solid-liquid separation, and washing and drying the resulting solid product to obtain the bleached fiber, where a method for the solid-liquid separation may be filtration; the reagent used for washing may be water, and specifically may be distilled water; and the drying may be air-dried at room temperature.

After obtaining the bleached fiber, the present disclosure involves mixing the bleached fiber with an alkaline solution for an alkaline treatment under ultrasonic and microwave conditions to obtain an alkali-treated slurry. The alkaline treatment under ultrasonic and microwave conditions according to the present disclosure may further improve the bleaching effect, enabling the whiteness of the resulting dietary fiber to reach a level of 80% or more, while maintaining the integrity of the fiber structure, functional properties such as water retention, texture and viscosity, fermentability, and nutritional quality.

As an embodiment of the present disclosure, the alkaline solution may include a NaOH solution or a Na₂CO₃ solution; the concentration of the NaOH solution may be 0.5-1 wt %, and specifically may be 0.5 wt %, 0.6 wt %, 0.7 wt %, 0.8 wt %, 0.9 wt % or 1 wt %; and the concentration of the Na₂CO₃ solution may be 1.5-2 wt %, and specifically may be 1.5 wt %, 1.6 wt %, 1.7 wt %, 1.8 wt %, 1.9 wt % or 2 wt %. As an embodiment of the present disclosure, the ratio of the bleached fiber to the amount of the alkaline solution may be 1-2 g:7.5-10 mL and specifically may be 1 g: 7.5 mL. As an embodiment of the present disclosure, the alkaline treatment is carried out under conditions including: an ultrasonic frequency that may be 60-90 kHz, and specifically may be 60 kHz, 65 kHz, 70 kHz, 75 kHz, 80 kHz, 85 kHz, or 90 kHz; a microwave power that may be 500-700 W, and specifically may be 500 W, 55 W, 600 W, 650 W, or 700 W; a temperature that may be 70-80° C., and specifically may be 70° C., 72° C., 75° C., 77° C., or 80° C.; and the duration of the alkaline treatment that may be 20-30 min, and specifically may be 20 min, 25 min, or 30 min. In an embodiment of the present disclosure, the alkaline treatment is carried out under the conditions described above; the cavitation phenomenon generated by ultrasonic waves may maximize the permeability of the alkaline solution, and simultaneously microwaves are used to maintain a stable temperature and increase chemical reactivity, thus enabling the effective removal of the lignin and other chromophoric compounds to ensure a better bleaching effect, and simultaneously avoiding damage to the integrity of the fiber structure; moreover, it also helps reduce reagent amount and energy consumption, enhancing the eco-friendliness of the preparation process.

In the present disclosure, after the alkaline treatment, post-processing is not required, and the alkali-treated slurry is directly mixed with aqueous hydrogen peroxide solution for an oxidation treatment to obtain dietary fiber. As an embodiment of the present disclosure, the concentration of the aqueous hydrogen peroxide solution may be 2-4 wt %, and specifically may be 2 wt %, 2.5 wt %, 3 wt %, 3.5 wt %, or 4 wt %; and the ratio of the amount of the bleached fiber to the amount of the aqueous hydrogen peroxide solution may be 40 g:280-320 mL, and specifically may be 40 g:300 mL. As an embodiment of the present disclosure, the oxidation treatment includes a first-stage oxidation treatment and a second-stage oxidation treatment in sequence. As an embodiment of the present disclosure, the first-stage oxidation treatment is carried out under ultrasonic and microwave conditions, the conditions of the first-stage oxidation treatment including: an ultrasonic frequency that may be 60-90 kHz, and specifically may be 60 kHz, 65 kHz, 70 kHz, 75 kHz, 80 kHz, 85 kHz, or 90 kHz; a microwave power that may be 500-700 W, and specifically may be 500 W, 550 W, 600 W, 650 W, or 700 W; a temperature that may be 40-70° C., and specifically may be 40° C., 50° C., 60° C., or 70° C.; and the duration of the first-stage oxidation treatment that may be 10-20 min, and specifically may be 10 min, 15 min, or 20 min. As an embodiment of the present disclosure, the second-stage oxidation treatment is carried out under conditions including: a temperature that may be 20-30° C., and specifically may be room temperature (25° C.); the duration of the second-stage oxidation treatment may be 1.5-2.5 h, and specifically may be 1.5 h, 2 h, or 2.5 h; and the second-stage oxidation treatment according to the present disclosure may be carried out in particular under static conditions, i.e., without stirring, ultrasound and microwave conditions, etc. The oxidation treatment carried out under the conditions described above in the embodiments of the present disclosure enables sufficient removal of residual lignin and other chromophoric compounds, ensures complete bleaching of the dietary fiber, and maintains the structural integrity of the dietary fiber, functional properties such as water retention, texture and viscosity, fermentability and nutritional quality, which is suitable for food-grade applications. Specifically, during the first-stage oxidation treatment, the generation of hydroxyl radicals (·OH) is promoted by ultrasonic cavitation, and simultaneously the temperature is maintained at 40-70° C. by controlling the microwave power, and the duration of the first-stage oxidation treatment is 10-20 min. Ultrasonically induced cavitation plays a critical role in the bleaching process, where locally generated bubbles rapidly collapse, producing intense shear forces and turbulence. These extreme localized conditions lead to the decomposition of hydrogen peroxide, resulting in the formation of highly reactive hydroxyl radicals (0H). These hydroxyl radicals will attack the lignin structure, promoting polymer decomposition.

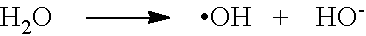

In addition, the uniform dielectric heating provided by the microwave system accelerates the decomposition of hydrogen peroxide, thereby increasing the overall reaction rate. This process also ensures that the structural integrity of the fiber is maintained while promoting efficient bleaching. The second-stage oxidation treatment is then continued at 20-30° C. for 1.5-2.5 h without stirring, ultrasound and microwave conditions. This extended incubation period may ensure complete oxidation and decomposition of lignin and other chromophores in the biomass but maintains glycosidic linkages.

As an embodiment of the present disclosure, after the oxidation treatment, the preparation method further includes subjecting the system obtained after the oxidation treatment to solid-liquid separation, and washing the resulting solid product, followed by freeze drying to obtain the dietary fiber, where a method for the solid-liquid separation may be filtration. The reagent used for washing may be water and specifically may be distilled water; and the conditions of the freeze drying are not specifically limited in the present disclosure, and any condition that is well known to the skilled in the art may be used.

In the present disclosure, first, laccase is used for delignification; then the bleaching treatment using hydrogen peroxide and an acid reagent (e.g., acetic acid) and assisted by ultrasound and microwaves allows the bleaching agent to penetrate more deeply into the fiber to ensure an excellent bleaching effect; then the alkaline treatment assisted by ultrasound and microwaves is used to enhance bleaching, followed by final whitening with hydrogen peroxide. The entire process may ensure effective decomposition and removal of lignin and sufficient bleaching of the fiber while maintaining the structural integrity of the fiber, functional properties such as water retention, texture and viscosity, fermentability and nutritional quality to a large extent. Furthermore, in the present disclosure, an aqueous solution containing hydrogen peroxide and an acid reagent (e.g., acetic acid) is used as a bleaching agent to ensure that the produced by-products are environmentally friendly and biodegradable, for example, hydrogen peroxide decomposes into water and oxygen, avoiding the use of toxic bleaching agents, thus minimizing potential environmental and health risks and simplifying the waste disposal process. In addition, the present disclosure employs a mild, multi-stage bleaching process, which may significantly reduce energy and reagent consumption while maintaining high production efficiency, making it suitable for industrial-scale production.

The present disclosure provides a dietary fiber prepared by the preparation method described in the aforementioned technical solution. As an embodiment of the present disclosure, dietary fiber is a bright white fiber, and the dietary fiber may have a CIE whiteness index of 77-86%, specifically 77.60%, 82.42% or 85.30%. In addition, the dietary fiber obtained by the present method exhibits preservation of arabinoxylan structure, evidenced by xylose content at least equal to, and typically greater than, that of untreated brewers' spent grain (>100% relative retention on a composition basis) while maintaining at least about 35-45% relative retention of arabinose. Consistent with this, linkage analysis further confirmed that the dietary fiber obtained by the present method retains key hemicellulose structures. 3,5-arabinofuranosyl residues were preserved at >85-90% relative to untreated brewers' spent grain, terminal arabinofuranosyl residues were essentially unchanged, and xylopyranosyl backbone linkages (4-Xylp) were maintained at >88-90%. By contrast, chlorine-bleached fibers exhibited extensive degradation of these linkages. This combined profile of high whiteness and hemicellulose structural integrity distinguishes the disclosed dietary fiber from fibers produced by conventional bleaching.

The dietary fiber has the following advantages:
(i) arabinose content of at least 18-20% and xylose content of at least 28-31% of total monosaccharides; and,
(ii) preservation of at least 85% of 3,5-arabinofuranosyl residues and at least 88% of 4-xylopyranosyl residues relative to untreated brewers' spent grain; wherein the fiber is characterized by both high whiteness and retention of arabinoxylan linkages not achieved by conventional bleach methods.

The present disclosure provides the use of the dietary fiber described in the aforementioned technical solution in food or nutrient products. The specific application mode of dietary fiber is not particularly limited in the present disclosure, and any mode that is well known to those skilled in the art may be used.

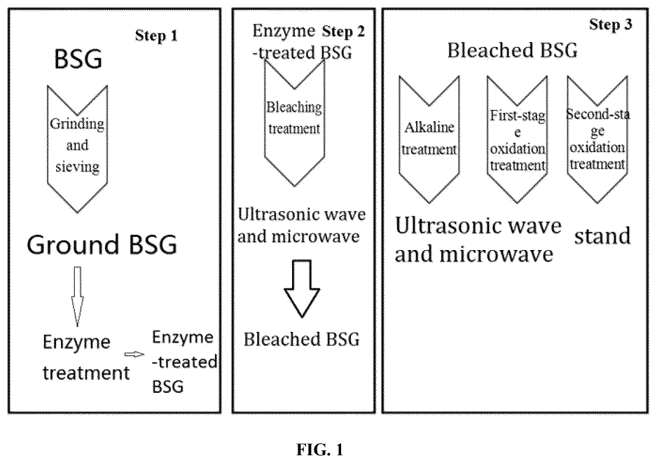

FIG. 1 shows a flow chart of the preparation of a dietary fiber in an embodiment of the present disclosure. The technical solutions of the present disclosure will be described clearly and completely below with reference to the examples of the present disclosure. Apparently, the examples described are merely some, rather than all of the examples of the present disclosure. Based on the examples in the present disclosure, all other examples obtained by those of ordinary skill in the art without involving creative effort belong to the scope of protection of the present disclosure.

The brewers' spent grain (BSG) used in the following experiments is supplied by China Resources Snow Breweries (Henan) Co., Ltd., which has a moisture content of 8.7 wt %; and the laccase is sourced from solarbio life sciences.

### Example 1

(1) BSG was dried at 80° C. for 12 h, and the resulting dried BSG was ground and sieved to obtain a BSG fiber having a particle size of 50-100 m. Laccase (200 U/g) was mixed with a sodium acetate buffer (pH=4.5) to obtain a laccase reagent with the concentration of laccase concentration being 14.5 U/g. The BSG fiber and the laccase reagent were mixed in a mass ratio of 13.33:1, and the resulting mixture was subjected to an enzyme treatment at 40° C. under stirring for 12 h and then filtered, and the filter cake was collected as an enzyme-treated BSG fiber.

(2) An aqueous hydrogen peroxide solution was mixed with acetic acid to obtain a bleaching agent, where in the bleaching agent, the concentration of hydrogen peroxide was 2 wt % and the concentration of acetic acid was 3 wt %. The enzyme-treated BSG fiber and the bleaching agent were mixed in a solid-to-liquid ratio of 40 g:300 mL, and bleached at a temperature of 70° C., an ultrasonic frequency of 70 kHz and a microwave power of 600 W for 10 min, followed by filtration. The filter cake was collected and washed with distilled water, followed by air-drying at room temperature to obtain a bleached BSG fiber.

(3) The bleached BSG fiber and a 0.8 wt % NaOH solution were mixed in a solid-to-liquid ratio of 40 g:300 mL, and subjected to an alkaline treatment at a temperature of 70° C., an ultrasonic frequency of 70 kHz and a microwave power of 600 W for 25 min. Then an aqueous hydrogen peroxide solution with a concentration of 3 wt % was added to the resulting alkali-treated slurry, with the solid-to-liquid ratio of the bleached BSG fiber to the aqueous hydrogen peroxide solution being 40 g:300 mL. The mixture was held at 70° C., an ultrasonic frequency of 70 kHz, and a microwave power of 600 W for 15 min, the ultrasonic wave and microwave were removed, and the system was left to stand at room temperature (25° C.) for 2 h and then filtered, and then the filter cake was collected and washed with distilled water to pH 7, followed by freeze drying to obtain a dietary fiber.

### Example 2

The operations were performed with reference to the method of Example 1, except that in step (1), the activity of laccase was 250 U/g, the concentration of the laccase reagent was 15.5 U/g, the temperature of the enzyme treatment was adjusted to 45° C., and the enzyme treatment time was reduced to 10 h; in step (2), during the bleaching treatment, the ultrasonic frequency was adjusted to 80 kHz, and the microwave power was adjusted to 700 W, and the bleaching treatment time was extended to 15 min; and in step (3), during the alkaline treatment, the concentration of the NaOH solution was adjusted to 1.0 wt %, the alkaline treatment time was extended to 30 min, the concentration of the hydrogen peroxide solution was adjusted to 4 wt %, and the holding time under the static condition was shortened to 1.5 h.

### Example 3

The operations were performed with reference to the method of Example 1, except that in step (1), the activity of laccase was 180 U/g, the concentration of the laccase reagent was 16 U/g, the temperature of the enzyme treatment was adjusted to 35° C., and the enzyme treatment time was extended to 14 h; in step (2), during the bleaching treatment, the ultrasonic frequency was adjusted to 60 kHz, and the microwave power was reduced to 500 W, and the bleaching treatment time was reduced to 8 min; and in step (3), during the alkaline treatment, the concentration of the NaOH solution was adjusted to 0.6 wt %, the alkaline treatment time was shortened to 20 min, the concentration of the hydrogen peroxide solution was adjusted to 2.5 wt %, and the holding time under the static condition was extended to 2.5 h.

### Comparative Example 1

The sample in this comparative example was specifically the enzyme-treated BSG fiber prepared in step (1) in Example 1.

### Comparative Example 2

The sample in this comparative example was specifically the bleached BSG fiber prepared in step (2) in Example 1.

### Comparative Example 3

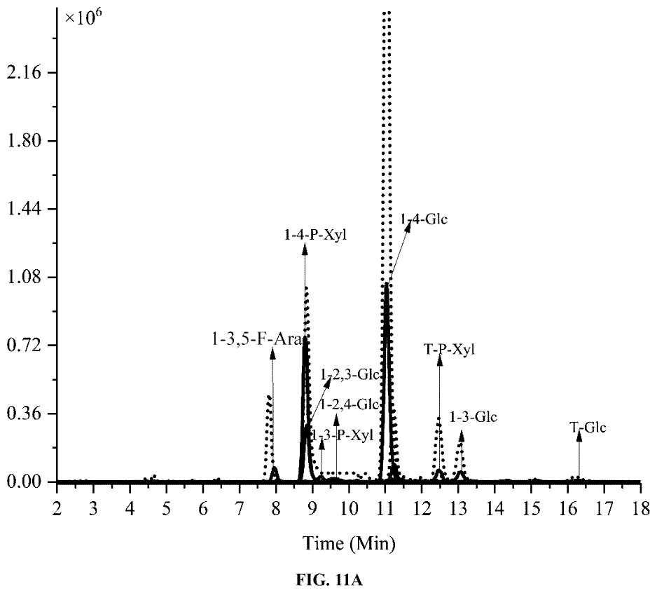

A chlorine-bleached fiber was prepared, including the following steps: 40 g of BSG was mixed with 200 mL of sodium chlorite (NaClO₂) solution (with a concentration of 0.05 g/mL), and the resulting mixture system was adjusted to pH 4 with hydrochloric acid (with a concentration of 100 mM). Bleaching treatment was carried out under stirring at a temperature of 80° C. for 7 h; and after the bleaching treatment, the mixture was filtered, and the filter cake was collected filter cake was collected and washed with distilled water to pH 7, followed by freeze drying to obtain the chlorine-bleached fiber. Significant degradation of arabinoxylan linkages was observed (loss of T-f-Ara, 2-f-Ara and 4-Xylp peaks in FIG. 10), whereas in the present method (Example 1), these linkages were largely retained, demonstrating unexpected structural preservation.

### Test Example 1

The products prepared in the examples and comparative examples were characterized and tested for performance and compared to untreated BSG. The details were as shown below:

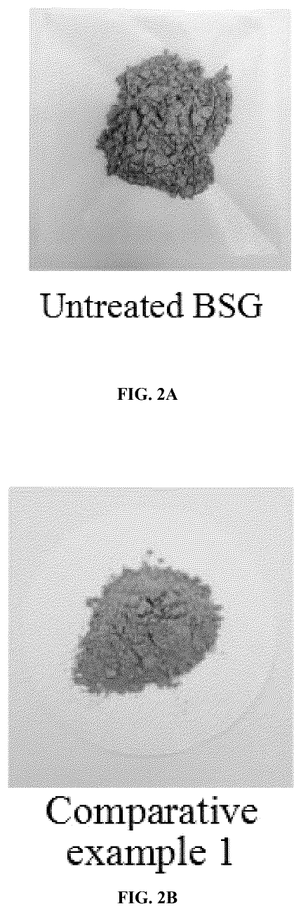

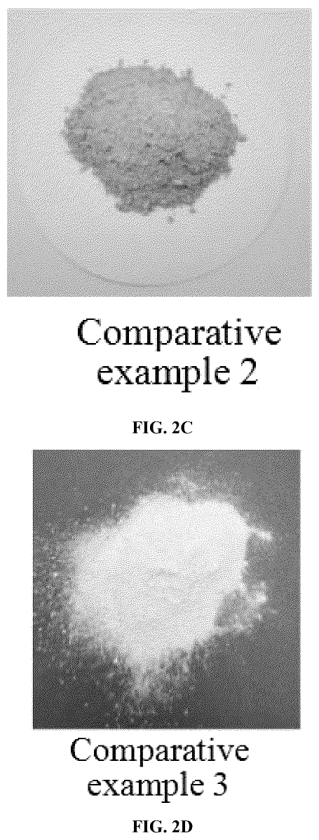

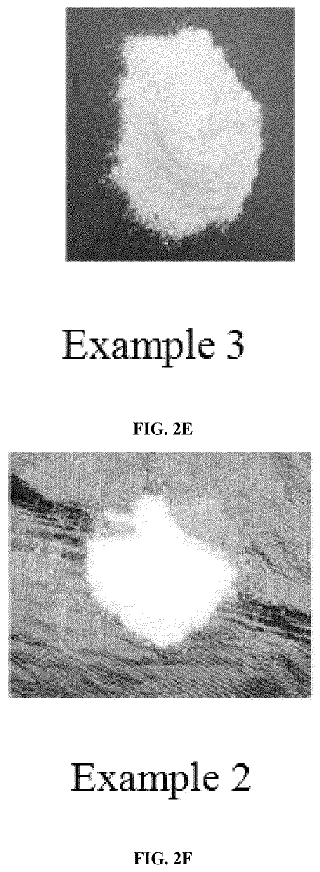

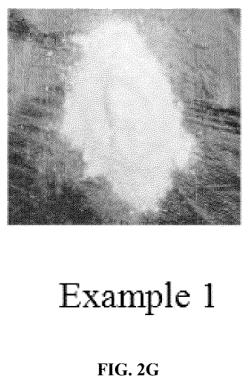

FIGS. 2A-2G show physical pictures of the products prepared in Examples 1-3 and Comparative examples 1-3. The results show that the dietary fibers prepared using the method according to the present disclosure have higher whiteness.

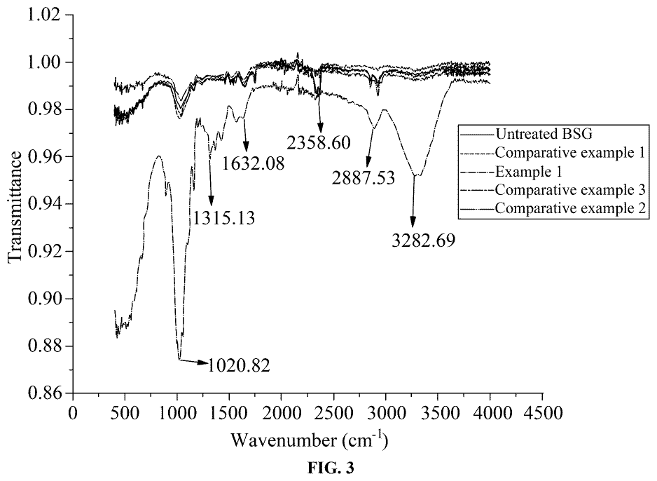

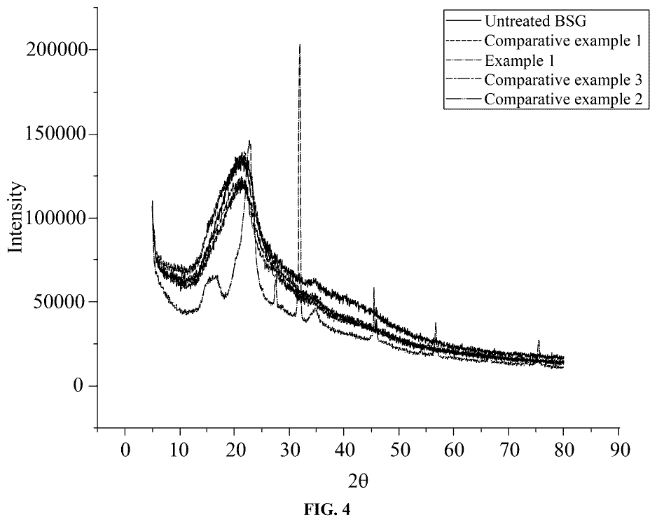

FIG. 3 shows Fourier-transform infrared (FTIR) spectra of the products prepared in Example 1 and Comparative examples 1-3. The difference in transmittance reflects the effectiveness of bleaching. The results show that the peaks at 1315 cm⁻¹, 1020 cm⁻¹, etc., are reduced, indicating an improvement in lignin removal in Example 1.

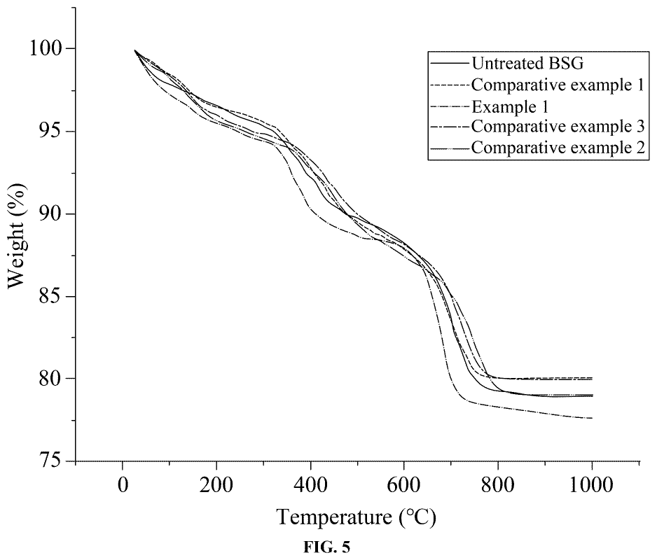

FIG. 4 shows X-ray diffraction (XRD) spectra of the products prepared in Example 1 and Comparative examples 1-3. The results show that the dietary fiber prepared in Example 1 exhibited reduced amorphous region intensity and enhanced crystalline peaks, indicating that the method of the present disclosure exhibits excellent delignification effects and is beneficial for improving the cellulose crystallinity.

FIG. 5 shows TGA curves of the products prepared in Example 1 and Comparative examples 1-3. The results show that the TGA curve of dietary fiber prepared in Example 1 shows less weight loss, indicating that lignin is removed, with more heat-stable cellulose being retained.

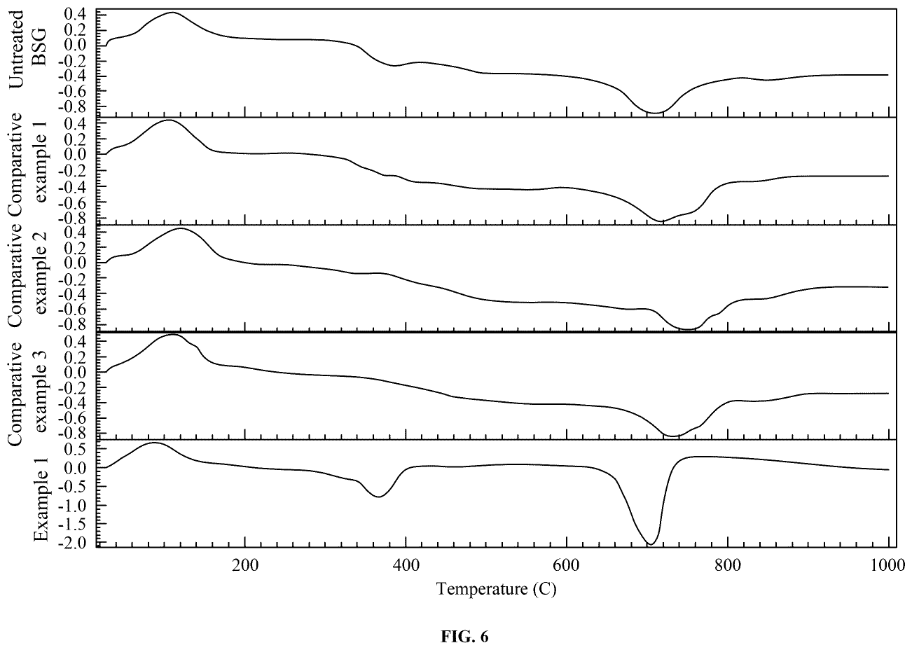

FIG. 6 shows the DSC curves of the products prepared in Example 1 and Comparative examples 1-3. The results show that the exothermic peaks at 200-400° C. on the DSC curve indicate the decomposition of lignin. Example 1 shows a decrease in intensity, indicating that lignin is effectively removed. The residual weight (600° C. or higher) represents the ash content. Example 1 shows slightly lower residues, due to effective delignification and impurity removal.

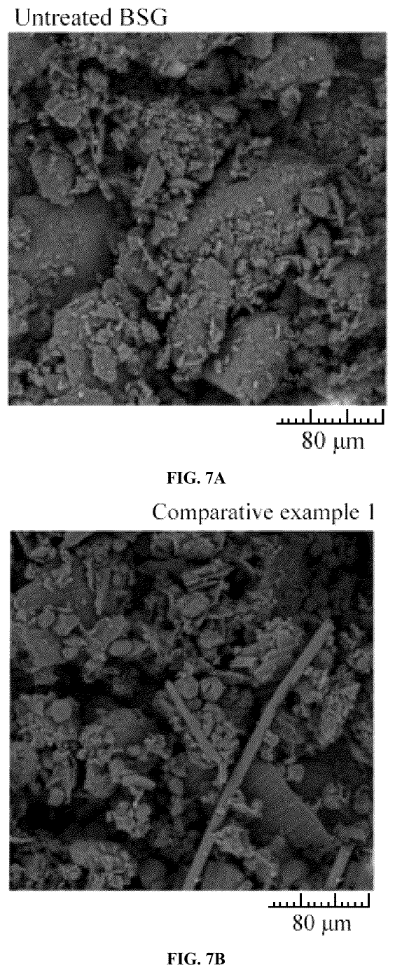

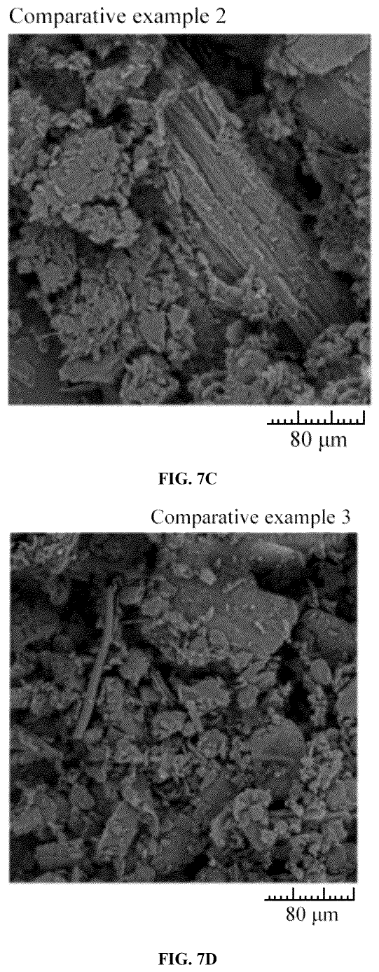

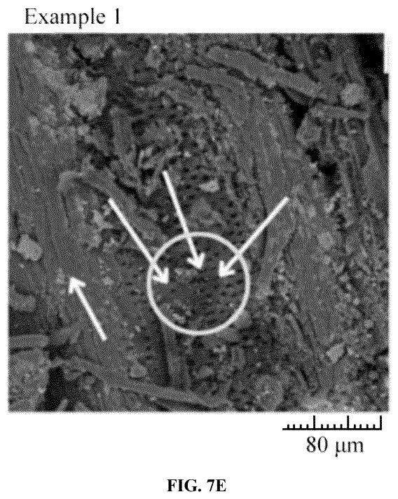

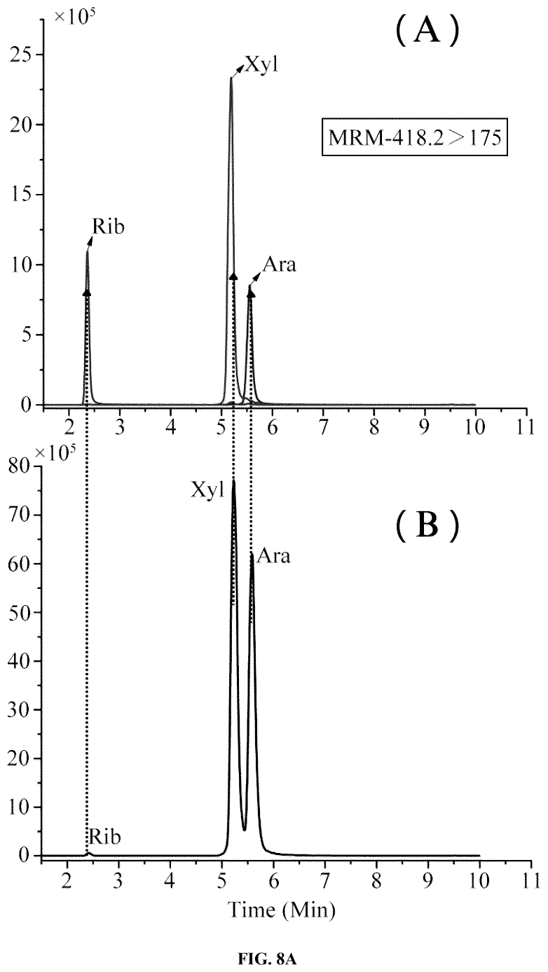

FIGS. 7A-7E show SEM images of the products prepared in Example 1 and Comparative Examples 1-3, and the results show that the surface of the product of Example 1 is smoother due to the removal of lignin, with more noticeable cracks and porosity. The internal structure of dietary fiber in Example 1 is better exposed, thereby improving water absorption and other functional properties.

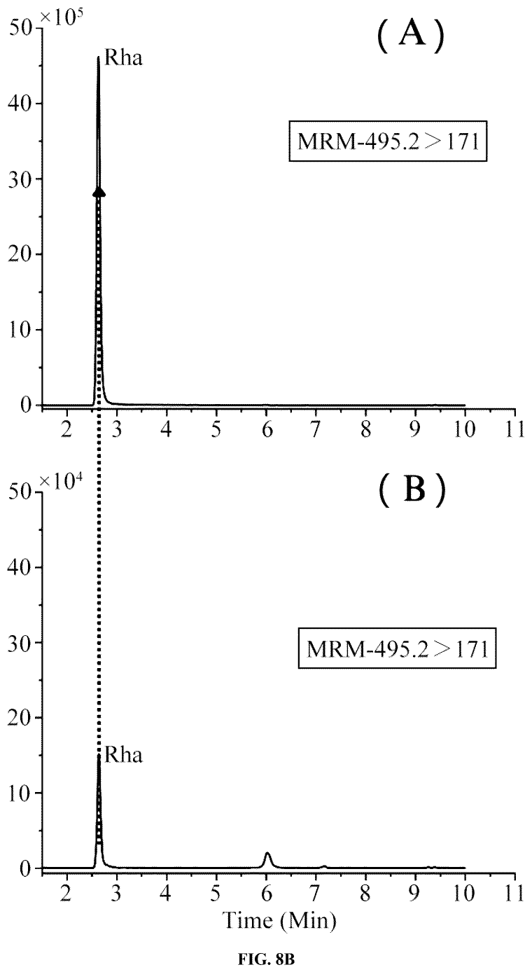

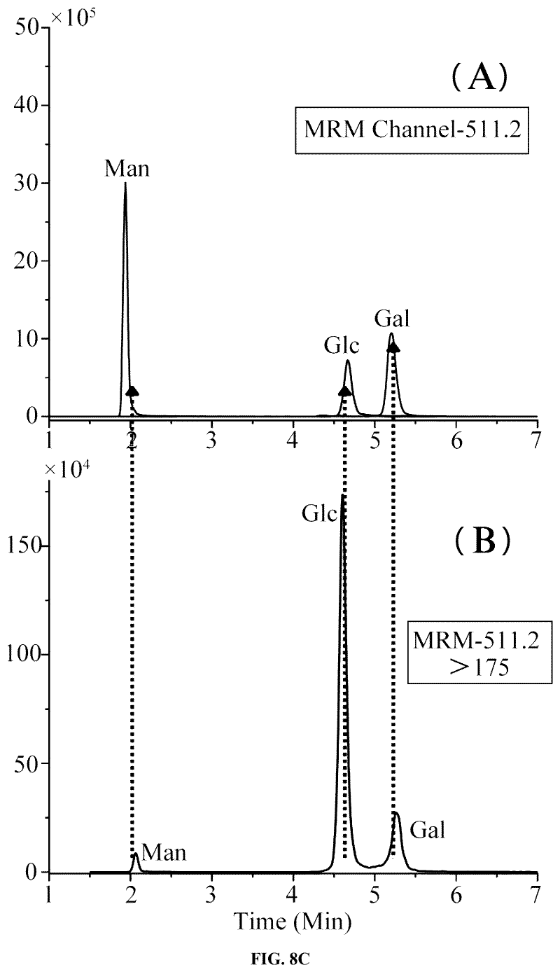

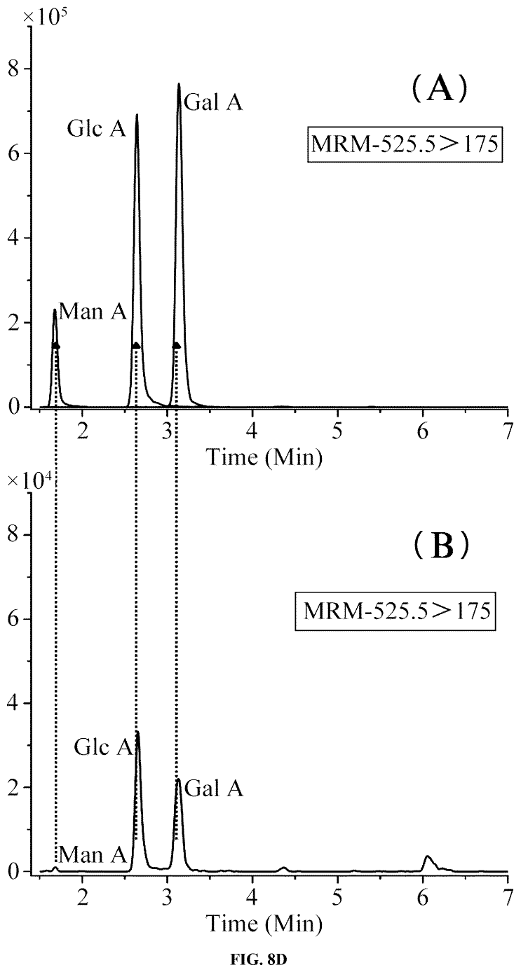

FIGS. 8A-8D are LC-MS/MS chromatograms illustrating the separation of standard monosaccharides [labelled as (A)] and example 1 monosaccharides [labelled as (B)], all identified in accordance with their retention times.

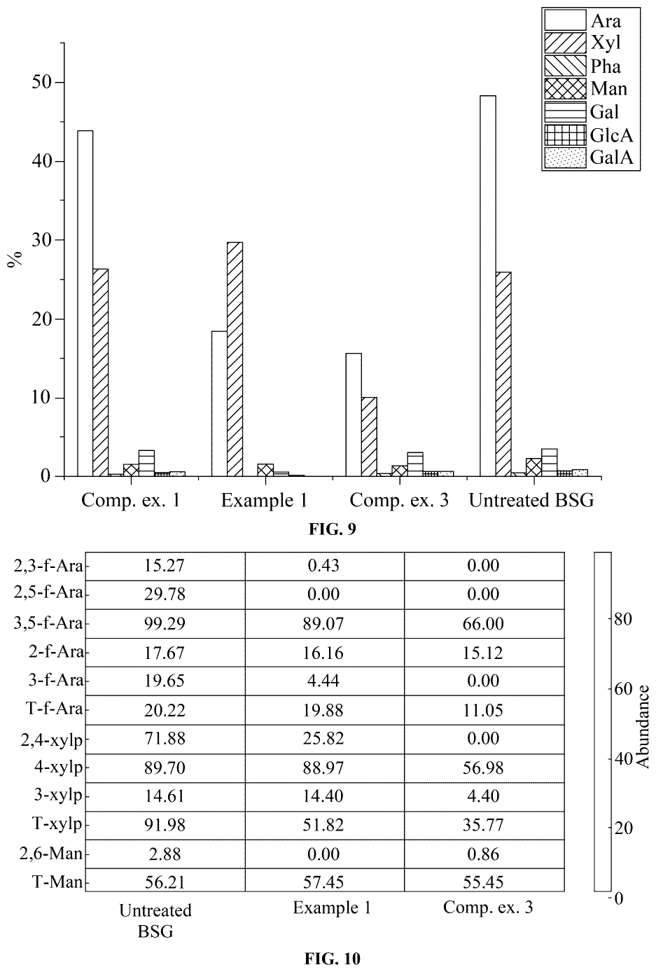

FIG. 9 shows Monosaccharide composition (%) of dietary fibers from brewer's spent grain subjected to different delignification treatments: enzymatic pretreatment alone (comparative example 1), example 1, comparative example 3, and untreated BSG fiber.

FIG. 10 shows differences in retention of major arabinoxylan glycosidic linkages in dietary fibers from brewer's spent grain (BSG) subjected to different delignification treatments: untreated BSG fiber, example 1 and comparative example 3.

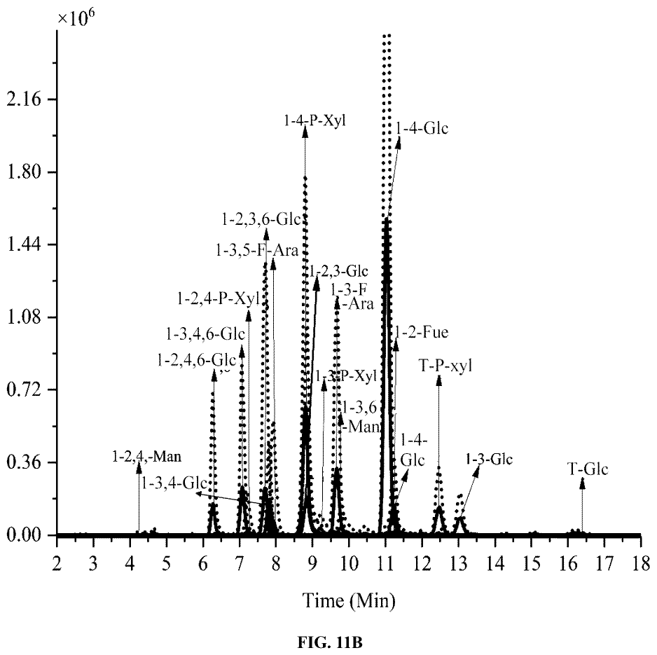

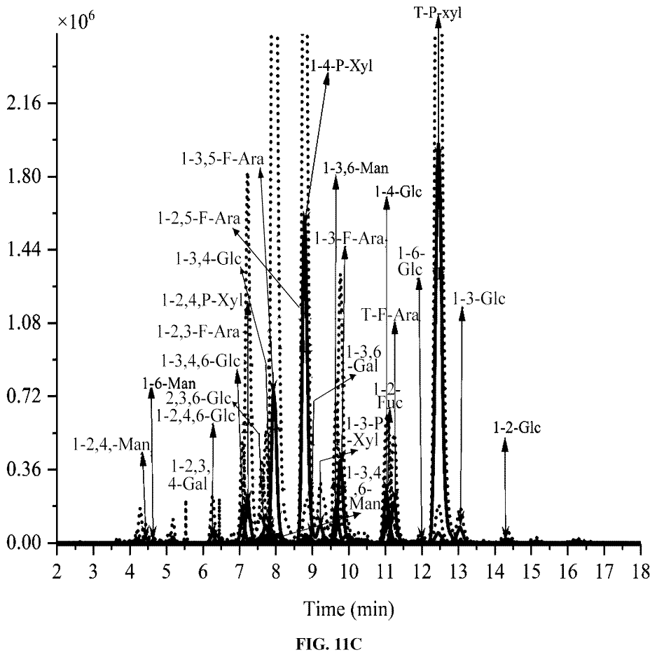

FIGS. 11A-11C show the MRM chromatograms showing linkage analysis for (A) Comparative example 3 (B) Example 1 and (C) Untreated BSG. The short dotted blue trace represents transitions of the quantifying ion at m/z 175.1, whereas the solid red and green traces represent transitions of qualifying ions at m/z 231.2 and m/z 217.1, respectively.

Table 1 shows the performance test results of the products prepared in Examples 1-3 and Comparative examples 1-3. As can be seen from Table 1, the dietary fiber prepared by the method of the present disclosure has high whiteness and excellent comprehensive performance.

TABLE 1
Performance test results of products prepared
in Examples 1-3 and Comparative examples 1-3

| | Example | Example | Example | Comparative | Comparative | Comparative |
|---|---|---|---|---|---|---|
| Test indicator | 1 | 2 | 3 | example 1 | example 2 | example 3 |
| CIE whiteness | 82.42 | 85.30 | 77.60 | 13.39 | 15.24 | 55.56 |
| index (%) | | | | | | |
| Klason lignin | 0.50 | 0.39 | 0.95 | 20.00 | 19.05 | 2.50 |
| (%) | | | | | | |
| Acid-soluble | 1.18 | 1.05 | 1.23 | 4.07 | 3.66 | 3.58 |
| lignin (%) | | | | | | |
| Total lignin (%) | 1.68 | 1.45 | 3.75 | 24.07 | 22.71 | 6.08 |
| Hemicellulose | 43.15 | 33.47 | 44.86 | 62.30 | 76.20 | 79.73 |
| (%) | | | | | | |
| Cellulose (%) | 56.65 | 66.33 | 54.94 | 37.50 | 23.60 | 20.07 |
| Water-binding | 4.64 | 3.81 | 4.58 | 3.27 | 3.52 | 3.04 |
| capacity (WBC, | | | | | | |
| g/g) | | | | | | |
| Swelling | 3.00 | 2.65 | 3.80 | 1.15 | 2.12 | 1.92 |
| capacity (SC, | | | | | | |
| g/g) | | | | | | |
| Binding | 0.54 | 0.51 | 0.54 | 0.79 | 0.72 | 0.51 |
| capacity (BC, | | | | | | |
| g/mL) | | | | | | |
| Oil holding | 3.16 | 2.40 | 3.57 | 1.68 | 2.24 | 4.08 |
| capacity (OHC, | | | | | | |
| g/g) | | | | | | |

The embodiments shown and described are merely exemplary and various alternatives, combinations, omissions, specific components, or foreseeable alternative components, understood by one having ordinary skill in the art, described in the present invention or within the field of the present invention, are intended to fall within the scope of the appending claims.

It will be appreciated that various aspects of the present invention and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the claims.

What is claimed is:

1. A method for preparing dietary fiber without using a chlorine-containing reagent and/or ozone, comprising the following steps:
mixing lignocellulosic biomass with a laccase reagent for an enzyme treatment to obtain an enzyme-treated fiber;
mixing the enzyme-treated fiber with a bleaching agent for a bleaching treatment under ultrasonic and microwave conditions to obtain a bleached fiber, wherein the bleaching agent is an aqueous solution containing hydrogen peroxide and an acid reagent;
mixing the bleached fiber with an alkaline solution for an alkaline treatment under ultrasonic and microwave conditions to obtain an alkali-treated slurry; and,
mixing the alkali-treated slurry with aqueous hydrogen peroxide solution for an oxidation treatment to obtain the dietary fiber.

2. The method of claim 1, wherein the lignocellulosic biomass comprises at least one selected from the group consisting of brewer's spent grain, sugar cane bagasse, rice hulls and straws.

3. The method of claim 1, wherein the laccase reagent comprises laccase and a buffer, the laccase has an activity of 180-250 U/g, and the buffer has a pH of 4.5-5.5; and concentration of the laccase in the laccase reagent is 13.5-16.5 U/g.

4. The method of claim 1, wherein the mass ratio of the lignocellulosic biomass to the laccase reagent is 13-14:1; and the enzyme treatment is carried out at a temperature of 35-50° C. for 6-14 h.

5. The method of claim 1, wherein concentration of hydrogen peroxide in the bleaching agent is 2-3 wt %, concentration of the acid reagent in the bleaching agent is 2-5 wt %, and the acid reagent is acetic acid; and a ratio of the enzyme-treated fiber to the bleaching agent is 40 g:280-320 mL; and the bleaching treatment is carried out under conditions comprising an ultrasonic frequency of 60-80 kHz, a microwave power of 500-700 W, a temperature of 40-70° C., and a duration of 8-20 min.

6. The method of claim 1, wherein the alkaline solution is selected from the group consisting of a NaOH solution and a Na₂CO₃ solution, concentration of the NaOH solution is 0.5-1 wt %, and concentration of the Na₂CO₃ solution is 1.5-2 wt %; a ratio of the bleached fiber to the alkaline solution is 1-2 g:7.5-10 mL; and the alkaline treatment is carried out under conditions comprising an ultrasonic frequency of 60-90 kHz, a microwave power of 500-700 W, a temperature of 70-80° C., and a duration of 20-30 min.

7. The method of claim 1, wherein concentration of the aqueous hydrogen peroxide solution is 24% wt %, and a ratio of the bleached fiber to the aqueous hydrogen peroxide solution is 40 g:280-320 mL; and the oxidation treatment comprises a first-stage oxidation treatment and a second-stage oxidation treatment in sequence; the first-stage oxidation treatment is carried out under ultrasonic and microwave conditions, conditions of the first-stage oxidation treatment comprising: an ultrasonic frequency of 60-90 kHz, a microwave power of 500-700 W, a temperature of 40-70° C., and a duration of 10-20 min; and the second-stage oxidation treatment is carried out without ultrasonic waves and microwaves, and the second-stage oxidation treatment is carried out under conditions of a temperature of 20-30° C., and a duration of 1.5-2.5 h.

8. A dietary fiber prepared by the preparation method of claim 1.

9. The dietary fiber of claim 8, wherein the dietary fiber has a CIE whiteness index of 77-86%.

10. A food or nutrient product, wherein comprising the dietary fiber of claim 8.

11. The method of claim 2, wherein the mass ratio of the lignocellulosic biomass to the laccase reagent is 13-14:1; and the enzyme treatment is carried out at a temperature of 35-50° C. for 6-14 h.

12. The method of claim 3, wherein the mass ratio of the lignocellulosic biomass to the laccase reagent is 13-14:1; and the enzyme treatment is carried out at a temperature of 35-50° C. for 6-14 h.

13. The food or nutrient product of claim 10, wherein the dietary fiber has a CIE whiteness index of 77-86%.
